(19) United States
(12) Patent Application Publication
(10) Pub. No.: US 2007/0281989 A1
(43) Pub. Date: Dec. 6, 2007

(54) Process for preparing duloxetine and intermediates thereof

(76) Inventors: Santiago Ini, Haifa (IL); Mili Abramov, Givataim (IL)

Correspondence Address: KENYON & KENYON LLP, ONE BROADWAY, NEW YORK, NY, 10004, US

(21) Appl. No.: 11/809,730

(22) Filed: May 31, 2007

Related U.S. Application Data
(60) Provisional application No. 60/809,977, filed on May 31, 2006.

(51) Int. Cl. A61K 31/381; C07D 333/22

(52) U.S. Cl. 514/438; 549/76

(57) ABSTRACT

Processes for preparing chemically pure duloxetine and chemically pure duloxetine intermediates are provided.

### CROSS-REFERENCE TO RELATED APPLICATIONS

The present application claims the benefit of the following U.S. Provisional Patent Application No. 60/809,977 filed May 31, 2006, the contents of which are incorporated herein by reference.

### FIELD OF THE INVENTION

The present invention relates to chemically pure duloxetine

### BACKGROUND OF THE INVENTION

Duloxetine HCl is a dual reuptake inhibitor of the neurotransmitters serotonin and norepinephrine. It is used for the treatment of stress urinary incontinence (SUI), depression, and pain management. It is commercially available as CYMBALTA®. Duloxetine hydrochloride has the chemical name (S)-(+)-N-methyl-3-(1-naphthalenyloxy)-3-(2-thienyl)propanamine hydrochloric acid salt and the following structure.

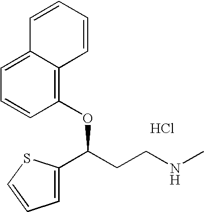

Duloxetine, as well as processes for its preparation, is disclosed in U.S. Pat. No. 5,023,269, EP Patent No. 457559, and U.S. Pat. No. 6,541,668.

The conversion of duloxetine to its hydrochloride salt is described in U.S. Pat. No. 5,491,243 and in Wheeler W. J., et al, J. Label. Cpds. Radiopharm, 1995, 36, 312. In both cases the reactions are performed in ethyl acetate.

Like any synthetic compound, duloxetine can contain extraneous compounds or impurities that can come from many sources. They can be unreacted starting materials, by-products of the reaction, products of side reactions, or degradation products. Impurities in duloxetine or any active pharmaceutical ingredient (API) are undesirable, and, in extreme cases, might even be harmful to a patient being treated with a dosage form of the API in which a sufficient amount of impurities is present. Furthermore, the undesired enantiomeric impurities reduce the level of the API available in the pharmaceutical composition.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and by-products of the reaction and adjunct reagents used in the reaction will, in most cases, also be present in the product mixture. At certain stages during processing of an API, it must be analyzed for purity, typically, by HPLC or TLC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use. In the United States, the Food and Drug Administration guidelines recommend that the amounts of some impurities be limited to less than 0.1 percent.

Generally, side products, by-products, and adjunct reagents (collectively “impurities”) are identified spectroscopically and/or with another physical method, and then associated with a peak position, such as that in a chromatogram or a spot on a TLC plate. (Strobel p. 953, Strobel, H. A.; Heineman, W. R., Chemical Instrumentation: A Systematic Approach, 3rd dd. (Wiley & Sons: New York 1989)). Thereafter, the impurity can be identified, e.g., by its relative position in the chromatogram, where the position in a chromatogram is conventionally measured in minutes between injection of the sample on the column and elution of the particular component through the detector. The relative position in the chromatogram is known as the “retention time.”

The retention time can vary about a mean value based upon the condition of the instrumentation, as well as many other factors. To mitigate the effects such variations have upon accurate identification of an impurity, practitioners use the “relative retention time” (“RRT”) to identify impurities. (Strobel p. 922). The RRT of an impurity is its retention time divided by the retention time of a reference marker. It may be advantageous to select a compound other than the API that is added to, or present in, the mixture in an amount sufficiently large to be detectable and sufficiently low as not to saturate the column, and to use that compound as the reference marker for determination of the RRT.

(+)-N-methyl-3-(1-naphtalenyloxy)-3-(3-thienyl)propanamine is disclosed by Olsen B. A et al, as an impurity obtained in the preparation of duloxetine (J. Lib. Chrom. & Rel. Technol, 1996, 19, 1993).

U.S. Pat. No. 4,956,388 discloses synthesis of N,N-dimethyl-3-(1-naphtalenyloxy)-3-(3-thienyl)propanamine and N-methyl-3-(1-naphtalenyloxy)-3-(3-thienyl)propanamine.

There is a need in the art for processes for preparation of duloxetine which are suitable for use on industrial scale and result in a product with high purity and.

### SUMMARY OF THE INVENTION

In one embodiment the present invention provides a process for preparing duloxetine (or a salt thereof) or a pharmaceutical composition thereof having less than about 2% by HPLC of N-methyl-3-(1-naphtalenyloxy)-3-(3-thienyl)propanamine (DLX-ISO3) comprising measuring level of the 3-acetyl thiophene in a batch of 2-acetyl thiophene, selecting a batch having less than about 2% of 3-acetyl thiophene; and synthesizing duloxetine (or a salt thereof) or a pharmaceutical composition thereof from the batch.

In another embodiment the present invention provides a process for preparing (+)-N,N-dimethyl-3-(1-naphtalenyloxy)-3-(2-thienyl)propanamine (DNT) having less than about 1% by HPLC of (+)-N,N-dimethyl-3-(1-naphtalenyloxy)-3-(3-thienyl)propanamine (DNT-ISO3) comprising measuring level of 3-acetyl thiophene in a batch of 2-acetyl thiophene, selecting a batch having less than about 2% of 3-acetyl thiophene; and preparing DNT or a salt thereof from the batch.

In another embodiment the present invention provides a process for preparing duloxetine (or a salt thereof) or a pharmaceutical composition thereof having less than about 1% by HPLC of N-methyl-3-(1-naphtalenyloxy)-3-(3-thienyl)propanamine (DLX-ISO3) comprising measuring level of DNT-ISO3 or a salt thereof in a batch of (+)-N,N-dimethyl-3-(1-naphtalenyloxy)-3-(2-thienyl)propanamine (DNT) or salt thereof, selecting a batch having less than about 1% of DNT-ISO3 or a salt thereof; and synthesizing duloxetine (or a salt) or a pharmaceutical composition thereof from the batch.

### DETAILED DESCRIPTION OF THE INVENTION

The present invention provides a process for preparing duloxetine substantially free of the impurity (+)-N-methyl-3-(1-naphtalenyloxy)-3-(3-thienyl)propanamine, referred to herein as DLX-ISO3, and represented by the formula:

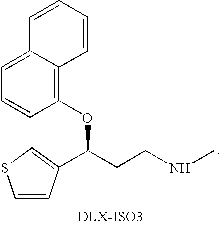

Also provided is a process for preparation of N,N-dimethyl-3-(1-naphthalenyloxy)-3-(2-thienyl)propanamine (DNT), an intermediate in the synthesis of duloxetine, substantially free of the impurity N,N-dimethyl-3-(1-naphtalenyloxy)-3-(3-thienyl)propanamine, referred to herein as DNT-ISO3.

Further provided is a process for preparation of a salt of N,N-dimethyl-3-(1-naphthalenyloxy)-3-(2-thienyl)propanamine, an intermediate in the synthesis of duloxetine, substantially free of the impurity that is the salt of N,N-dimethyl-3-(1-naphtalenyloxy)-3-(3-thienyl)propanamine, referred to herein as DNT-ISO3 salt. Preferred salts are: maleate, succinate, fumarate, benzensulfonate and Di-P-toluoyl-L-tartrate. Most preferably, the salt is a maleate salt.

We have found that batches of the starting material in the synthesis of duloxetine, specifically those of 2-acetylthiophene, are contaminated with the impurity 3-acetylthiophene. Further, at each step in the synthesis of duloxetine, this impurity is also transformed. By detecting and controlling amount of this impurity in the beginning of the synthetic process, we have found that it is possible to eliminate or reduce the corresponding 3-thienyl impurities from being present in upstream intermediates and products.

Preferably the batches of 2-acetylthiophene contain less than about 2%, more preferable less than about 1% and most preferably less than about 0.5% by HPLC of 3-acetylthiophene. In one embodiment, a batch having about 0.56% of the impurity is chosen.

Use of these batches for synthesis results in duloxetine and its pharmaceutical compositions, particularly tablets, being substantially free of DLX-ISO3. As used herein, and with reference to duloxetine, substantially free means containing less than about 2% DLX-ISO3, as measured by HPLC. Preferably duloxetine contains less than about 0.5%, more preferably less than about 0.14%, even more preferably less than about 0.07% and even more preferably, less than about 0.04%, and most preferably below the detection limit; i.e., the duloxetine contains essentially 0.0 percent DLX-ISO3 within the error limits of the detection of HPLC.

Use of these batches for synthesis also results in DNT or its salt being substantially free of DNT-ISO3 or its salt. As used herein, and with reference to DNT, substantially free means containing less than about 1% DNT-ISO3, as measured by HPLC, preferably less than about 0.5%, even more preferably about 0.14%, even more preferably less than about 0.07% and even more preferably, less than about 0.04%, and most preferably below the detection limit; i.e., the DNT or its salt contains essentially 0.0 percent DNT-ISO3 within the error limits of the detection of HPLC. Preferably, the pure DNT is (S)-DNT. Preferred salts are: maleate, succinate, fumarate, benzensulfonate and Di-P-toluoyl-L-tartrate. Most preferably the DNT salt is DNT maleate.

After selecting a desirable batch of 2-acetyl thiophene, duloxetine is synthesized. The synthesis generally comprises reacting 2-acetylthiophene with paraformaldehyde and dimethylamine, or a salt thereof, reduction with a reducing agent, such as sodium borohydride, chiral resolution with mandelic acid, reaction with a halonaphtalene and reaction with maleic acid.

In another embodiment, a batch of DNT is selected. Preferably the batch contains less than about 0.5% of DNT-ISO3 or salt thereof, more preferably less than about 0.14% of DNT-ISO3 or salt thereof and most preferably about 0.0% of DNT-ISO3 or salt thereof.

A general scheme for the synthesis of DNT (or salts) and duloxetine (or salts) is as follows:

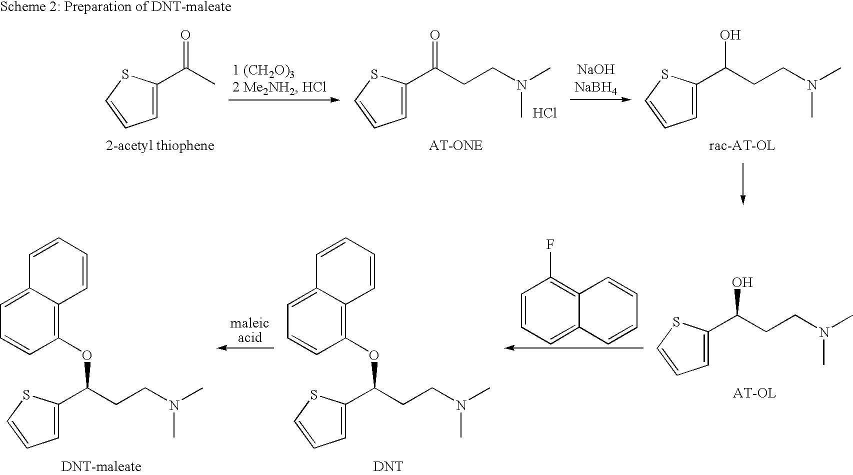

More specifically, the synthesis can comprise:
1) combining 2-acetylthiophene, paraformaldehyde, dimethylamine and a solvent to obtain a mixture containing 3-dimethylamino-1-(2-thienyl)-1-propanone (AT-ONE);
2) combining the mixture with a strong base, reducing agent and a C₁-C₈ alcohol or a mixture of C₁-C₈ alcohol with water to obtain a racemic mixture of N,N-dimethyl-3-(2-thienyl)-3-hydroxypropanamine (AT-OL);
3) combining the racemic mixture of AT-OL with mandelic acid in a solvent selected from the group consisting of: water, C₁₋₈ alcohols, C₃₋₈ ketones, C₂₋₈ alkyl esters, C₅₋₈ aromatic hydrocarbons, and mixtures thereof to obtain enantiomerically pure AT-OL;
4) combining the enantiomerically pure AT-OL with halonaphthalene and a base to obtain DNT;
5) converting the obtained DNT to a DNT salt, such as the maleate. Processes for preparation of duloxetine are also disclosed in US2006/0194869 and US2006/027073 1, incorporated herein by reference.

The dimethylamine used can be introduced into the reaction mixture either in its based form, or as a salt. Preferably, the dimethylamine is dimethylamine HCl.

The solvent used in step (a) may be any inert solvent. Typically, polar organic solvent can be used. Preferably, C₁-C₈ alcohol are used, most preferably, the solvent is isopropyl alcohol (IPA).

Preferably, the combination of 2-acetylthiophene, paraformaldehyde source, dimethylamine and the solvent is heated to obtain the mixture containing AT-ONE. More preferably, the combination is heated to reflux.

Typically, the mixture containing AT-ONE is filtrated, to obtain a solid, and further combined with a strong base, sodium borohydride and a polar aprotic solvent.

Preferably, the strong base is selected from the group consisting of alkali metal hydroxide and alkali metal alkoxides. More preferably, the strong base is potassium hydroxide (KOH), sodium methoxide, or sodium hydroxide (NaOH).

The strong base may be added portionwise in order to increase the chemical yield.

Typically, the strong base is combined with a solution of AT-ONE in the solvent. Preferably, the solution is cooled prior to the addition of the base.

In one specific embodiment, a solution of AT-ONE in methanol and water is cooled to a temperature of about 0° C. and further combined with sodium hydroxide.

Preferably, the reducing agent is selected from the group consisting of: sodium borohydride (NaBH₄), lithium borohydride (LiBH₄), lithium aluminum hydride (LiAlH) and selectride. More preferably, the reducing agent is NaBH₄.

The mixture containing AT-OL obtained, after combining with the reducing agent, is a racemic mixture, which is further subjected to chiral resolution.

Preferably, the organic solvent used for the chiral resolution is selected from the group consisting of isopropanol, methyl iso-butyl ketone, and toluene.

Combining of the racemic mixture of AT-OL, mandelic acid and the solvent can be carried out at a temperature of about room temperature to about reflux temperature. Preferably, racemic AT-OL is combined with mandelic acid in the solvent at a temperature of about 50° C.

The reaction mixture may be further heated to accelerate the chiral resolution process. Preferably, the heated reaction mixture is maintained after a precipitate appears, more preferably for about 45 minutes.

Preferably, the heated reaction mixture is cooled to a temperature of about 15° C. to about 25° C., to obtain a precipitate.

The obtained enantiomerically pure AT-OL can be either (S)-AT-OL or (R)-AT-OL, depending on the enantiomerically pure acid introduced into the reaction. For example, when (S)-mandelic acid is used, (S)-AT-OL is obtained.

The halonaphthalene is preferably 1-fluoronaphthalene or 1-chloronaphthalene.

In one specific embodiment, DNT is prepared by providing a solution of a base selected from the group consisting of: alkali metal hydroxide, sodium and alkali metal alkoxides, AT-OL and polar aprotic solvent at a temperature of from about 15° C. to about the reflux temperature of the solvent; combining the solution with 1-fluoronaphthalene or 1-chloronaphthalene, with or without a phase transfer catalyst, to obtain a mixture; heating the mixture to a temperature of from about room temperature to about the reflux temperature of the solvent and recovering DNT.

The DNT may be converted to a salt of DNT by a process comprising combining DNT and the respective acid to obtain the desired salt. Preferred salts are: maleate, succinate, fumarate, benzensulfonate and Di-P-toluoyl-L-tartrate. Nost preferably, the salt is a maleate salt, and the acid is maleic acid.

In one embodiment, the process comprises combining with maleic acid a solution of DNT in at least one solvent to obtain a precipitate of DNT-maleate; and recovering the DNT-maleate. The maleic acid may be either added as a solid or as a solution or suspension in an organic solvent. The solvent is preferably selected from C₁₋₈ alcohols, C₃₋₇ esters, C₃₋₈ ethers, C₃₋₇ ketones, C₆₋₁₂ aromatic hydrocarbons, acetonitrile, and water. More preferably, the solvent is acetone, n-butanol, ethyl acetate, methyl tert-butyl ether, toluene or water. Most preferably, the solvent is ethyl acetate, acetone, or n-butanol.

Typically, the combination of DNT, maleic acid, and solvent is heated. Preferably, the combination is heated to about reflux temperature of the solvent. Preferably, the combination is maintained, while heating, for about 15 minutes.

Preferably, the combination is cooled to induce precipitation of the DNT-maleate. More preferably, the combination is cooled to a temperature of about 15° C. Preferably, the combination is maintained, while cooled, for about 20 minutes to about 5 days to induce precipitation of the DNT-maleate.

The DNT maleate prepared according to the above process may be recovered by any method known in the art, such as separating the phases, and concentrating the organic phase until a dry residue is formed. Prior to separation, the DNT may be washed in order to remove inorganic impurities, or organic impurities that are miscible in water.

The DNT salt obtained, such as the maleate, can be converted to duloxetine by subjecting the DNT salt to basic hydrolysis. This process can comprise demethylation of the DNT with alkyl chlorofornate, followed by basic hydrolysis.

In one embodiment the conversion of DNT to duloxetine is performed as described in U.S. Pat. No. 5,023,269 or in U.S. publication No. 2006/0194869. Preferably, the conversion is performed by a process comprising: dissolving DNT in an organic solvent to obtain a solution; combining the solution with an alkyl haloformate to obtain duloxetine alkyl carbamate; and combining the duloxetine alkyl carbamate with an organic solvent and a base to obtain duloxetine. More preferably, the conversion is performed by a process comprising dissolving DNT in a water immiscible organic solvent to obtain a first solution; adding alkyl chloroformate to the first solution at a temperature of about 5° C. to less than about 80° C. to obtain duloxetine alkyl carbamate; combining the duloxetine alkyl carbamate with an organic solvent and a base to obtain a mixture; heating the mixture to reflux temperature and maintaining the mixture at reflux temperature for at least 1 to 3 hours; cooling the mixture and adding water and an additional amount of an organic solvent to the mixture to obtain duloxetine.

If a commercially available batch does not meet the purity requirements for selection, it may be possible to improve the purity level before use in the synthetic process. For example, if the measured 2-acetylthiophene batch contains more than about 2% of 3-acetylthiophene, it may be purified according to, e.g., the process described in U.S. Pat. No. 5,371,240, incorporated herein by refernce.

Additionally, if the measured DNT batch contains more than about 1% of the DNT-ISO3 impurity, it may be purified by converting it to a salt of DNT, and basifying the obtained salt to obtain DNT, substantially as described in examples 6 and 7 below for the maleate salt.

Similarly, if the measured DNT- salt batch contains more than about 1% of the DNT-ISO3 salt impurity, it may be purified by basifying to obtain DNT, followed by converting the obtained DNT to the DNT salt. Most preferably, the salt is a maleate salt.

These steps may be repeated in order to decrease the impurities content even more.

Having described the invention with reference to certain preferred embodiments, other embodiments will become apparent to one skilled in the art from consideration of the specification. The invention is further defined by reference to the following examples, describing in detail the analysis of the duloxetine HCl and methods for preparing the duloxetine HCl of the invention.

It will be apparent to those skilled in the art that many modifications, both to materials and methods, may be practiced without departing from the scope of the invention.

### EXAMPLES

### HPLC Method for Measuring Chemical Purity

| Column: | Hypersyl Gold (150 × 4.6 5μ) |
|---|---|
| Mobile phase: | (A) 63% ((NH₄)H₂PO₄ (0.02M) pH-2.5): 37% |
| | (78% MeOH:22% THF) |
| | (B) 20% ((NH₄)H₂PO₄ (0.02M) pH-2.5): 80% ACN |
| Gradient: | From 0 to 15 min (A) isocraticaly |
| | From 15 to 60 min (B) increases from 0 to 75% |
| Detection: | 230 nm |
| Flow: | 1 ml/min |
| Detection limit: | 0.02% |

### Example 1

### Preparation of AT-ONE

A mixture of 50 g of 2-acetylthiophene (containing 0.56% 3-acetylthiophene), 42 g of dimethylamine hydrochloride, 18 g of paraformaldhyde, and 2 g of HCl [32%] in 125 ml IPA were heated to reflux for 4 hours. The mixture was cooled to 0° C., and the resulting solid was collected by filtration, washed with ethanol (125 ml×2), and used in the next step without further action.

### Example 2

### Preparation of Rac-AT-OL

A solution of 90 g of AT-ONE from the previous example in 290 ml of methanol and 145 ml of water was cooled to 0° C. and 14 ml of NaOH [47%] were gradually added till pH 10. To the resulting solution was added portion added 12.1 g of sodium borohydride, and the mixture was allowed to warm to room temperature overnight. The methanol was evaporated under reduced pressure, and 250 ml were added, followed by the slow addition of concentrated HCl till pH 1.5, and stirred for an additional 20 minutes.

### Example 3

### Preparation of AT-OL-Mandelate

After basification with NaOH, the phases were separated, the water phase was washed with MTBE, and the combined organic phases were washed with brine. To the MTBE solution was added a solution of 16.4 g of (S)-mandelic acid in 40 ml ethanol, the resulting mixture was stirred at reflux for 1.25 hours, and then cooled to room temperature. The resulting solid was filtered, washed with MTBE, and dried in a vacuum oven to give 25 g of (S)-AT-OL mandelate.

### Example 4

### Preparation of AT-OL

To 20 g of AT-OL-mandelate in a mixture of 60 ml water and 90 ml MTBE were added NaOH [47%] till pH 9, and stirred at room temperature. After 30 minutes, the phases were separated, the organic phases were washed with water, and the residue evaporated to dryness.

### Example 5

### Preparation of DNT

To a solution of 7 g. of AT-OL in 42 ml of DMSO at room temperature were added 5 g of KOH, and stirred for an additional time. After 1 hour, 5 ml of 1-fluoronaphthalene were added, the solution was heated to 60° C., and stirred overnight.

To the reaction mixture was added water, followed by 80 ml HCl [5%], and extracted with 40 ml ethyl acetate (twice). After phase separation, the organic phase was washed with brine, and concentrated to dryness to give 10.5 g of brownish oil containing 0.12% of DNT-ISO3: 0.12%.

### Example 6

### Preparation of DNT-Maleate Free of DNT-ISO3

3.8 g of maleic acid were added to a solution of 10 g of DNT-base dissolved in 100 ml of ethyl acetate heated to reflux and cooled to room temperature. The resulting solid was filtered and washed with ethyl acetate. After drying in a vacuum oven at 50° C. for 16 hours, 5.5 g of DNT-maleate were obtained free of DNT-ISO3.

### Example 7

### Preparation of DNT Base Free of DNT-ISO3

A 2 liter reactor equipped with a mechanical stirrer is charged with a mixture of 107 g DNT-Maleate, 600 ml of water, 96 ml of a solution of ammonium hydroxide [22%], and 1 liter toluene. The mixture is stirred at 25° C. for 20-30 minutes, and the organic phase separated and washed with water (3×300 ml). The toluene solution containing the DNT-base free of DNT-ISO3 is evaporated to dryness.

### Example 8

### Preparation of (S)-Duloxetine Ethyl Carbamate

A 1 liter reactor, equipped with a mechanical stirrer, thermometer, dean stark, and condenser, is charged with (S)-DNT-base obtained in Example 6 dissolved in 1020 ml of toluene and 13 g of K₂CO₃. The mixture is heated, and an azeotropic distillation of 284 ml of the mixture is performed. After cooling to 50° C., 47.46 ml of ethyl chloroformate are added over a period of a half hour, and the reaction mixture is stirred at the same temperature for an additional 2 hours. After cooling to room temperature, the reaction mixture is washed with 230 ml of water, 130 ml of a 5 percent HCl solution, 130 ml of water, 130 ml of a 5 percent NaHCO₃ solution, and 130 ml of water. The resulting toluene solution of (S)-duloxetine ethyl carbamate is used in Example 9 without evaporation.

### Example 9

### Preparation of (S)-Duloxetine Base Free of DLX-ISO3

A 1 liter reactor, equipped with a mechanical stirrer, thermometer, and condenser, is charged with the solution of (S)-duloxetine ethyl carbamate in toluene prepared in Example 7. The mixture is heated, and an azeotropic distillation of 268 ml is performed. After cooling to 60° C., 82.18 g of an 85 percent KOH solution are added, and the mixture is heated to 94° C. for about 4 hours. After cooling to 60° C., 270 ml of water are added, and the resulting organic phase is washed three times with 270 ml of water, and treated with 4.6 g of charcoal (SX1) for 15 minutes, filtrated through a hyperflow bed, and washed with 60 ml of toluene. The solution is distillated at 30° to 40° C. under a vacuum of 20 to 30 mmHg until a volume of about 1 to 2 volumes of toluene is obtained.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

What is claimed is:

1. A process for preparing duloxetine (or a salt thereof) or a pharmaceutical composition thereof having less than about 2% by HPLC of N-methyl-3-(1-naphtalenyloxy)-3-(3-thienyl)propanamine (DLX-ISO3) comprising measuring level of the 3-acetyl thiophene in a batch of 2-acetyl thiophene, selecting a batch having less than about 2% of 3-acetyl thiophene; and synthesizing duloxetine (or a salt thereof) or a pharmaceutical composition thereof from the batch.

2. The process of claim 1, wherein the batch contains less than about 1% of 3-acetyl thiophene.

3. The process of claim 2, wherein the batch contains less than about 0.5% of 3-acetyl thiophene.

4. The process of claim 1, wherein the batch contains less than about 0.56% of 3-acetyl thiophene.

5. The process of claim 1, wherein the duloxetine or its composition contains less than about 0.5% of DLX-ISO3.

6. The process of claim 5, wherein the duloxetine or its composition contains less than about 0.14% of DLX-ISO3.

7. The process of claim 6, wherein the duloxetine or its composition contains about 0.0% of DLX-ISO3.

8. The process of claim 1 wherein the synthesis is carried out by reacting 2-acetylthiophene with paraformaldehyde and base to obtain 3-dimethylamino-1-(2-thienyl)-1-propanone (AT-ONE), reducing AT-ONE to obtain N,N-dimethyl-3-(2-thienyl)-3-hydroxypropanamine (AT-OL), resolving AT-OL, reacting the AT-OL with halonaphtalene to obtain (+)-N,N-dimethyl-3-(1-naphtalenyloxy)-3-(2-thienyl)propanamine (DNT), and hydrolyzing the DNT to obtain duloxetine.

9. The process of claim 8, further comprising reacting the DNT with maleic acid.

10. The process of claim 8, further comprising reacting the duloxetine with HCl to obtain duloxetine HCl.

11. The process of claim 8, wherein the base is dimethylamine.

12. The process of claim 8, wherein the reducing agent is NaBH₄.

13. The process of claim 8, wherein the halonaphtalene is 1-fluoronaphthalene or 1-chloronaphthalene.

14. The process of claim 8, wherein the hydrolysis is carried out by reacting DNT with an alkyl haloformate to obtain a carbamate, and combining the carbamate with a base.

15. A process for preparing (+)-N,N-dimethyl-3-(1-naphtalenyloxy)-3-(2-thienyl)propanamine (DNT) having less than about 1% by HPLC of (+)-N,N-dimethyl-3-(1-naphtalenyloxy)-3-(3-thienyl)propanamine (DNT-ISO3) comprising measuring level of 3-acetyl thiophene in a batch of 2-acetyl thiophene, selecting a batch having less than about 2% of 3-acetyl thiophene; and preparing DNT or a salt thereof from the batch.

16. The process of claim 15, wherein the batch contains less than about 1% of 3-acetyl thiophene.

17. The process of claim 16, wherein the batch contains less than about 0.5% of 3-acetyl thiophene.

18. The process of claim 15, wherein the batch contains less than about 0.56% of 3-acetyl thiophene.

19. The process of claim 15, wherein the DNT contains less than about 0.5% of DNT-ISO3.

20. The process of claim 19, wherein the DNT contains less than about 0.14% of DNT-ISO3.

21. The process of claim 20, wherein the duloxetine or its composition contains about 0.0% of DNT-ISO3.

22. The process of claim 15 wherein the synthesis is carried out by reacting 2-acetylthiophene with paraformaldehyde and base to obtain 3-dimethylamino-1-(2-thienyl)-1-propanone (AT-ONE), reducing AT-ONE to obtain N,N-dimethyl-3-(2-thienyl)-3-hydroxypropanamine (AT-OL), resolving AT-OL, and reacting the AT-OL with halonaphtalene to obtain (+)-N,N-dimethyl-3-(1-naphtalenyloxy)-3-(2-thienyl)propanamine (DNT).

23. The process of claim 22, further comprising reacting the DNT with maleic acid.

24. The process of claim 22, wherein the base is dimethylamine.

25. The process of claim 22, wherein the reducing agent is NaBH₄.

26. The process of claim 22, wherein the halonaphtalene is 1-fluoronaphthalene or 1-chloronaphthalene.

27. A process for preparing duloxetine (or a salt thereof) or a pharmaceutical composition thereof having less than about 1% by HPLC of N-methyl-3-(1-naphtalenyloxy)-3-(3-thienyl) propanamine (DLX-ISO3) comprising measuring level of DNT-ISO3 or a salt thereof in a batch of (+)-NN-dimethyl-3-(1-naphtalenyloxy)-3-(2-thienyl)propanamine (DNT) or salt thereof, selecting a batch having less than about 1% of DNT-ISO3 or a salt thereof; and synthesizing duloxetine (or a salt) or a pharmaceutical composition thereof from the batch.

28. The process of claim 27, wherein the batch contains less than about 0.5% of DNT-ISO3 or salt thereof.

29. The process of claim 28, wherein the batch contains less than about 0.14% of DNT-ISO3 or salt thereof.

30. The process of claim 29, wherein the batch contains about 0.0% of DNT-ISO3 or salt thereof.

31. The process of claim 27, wherein the duloxetine or its composition contains less than about 0.5% of DLX-ISO3.

32. The process of claim 31, wherein the duloxetine or its composition contains less than about 0.14% of DLX-ISO3.

33. The process of claim 32, wherein the duloxetine or its composition contains about 0.0% of DLX-ISO3.

34. The process of claim 27, wherein the DNT salt is a maleate salt.

35. The process of claim 27, wherein the DNT-ISO3 salt is a maleate salt.

36. The process of claim 27, wherein DNT is converted to duloxetine by hydrolysis.

37. The process of claim 36, wherein the hydrolysis is carried out by reacting DNT with an alkyl haloformate to obtain a carbamate, and combining the carbamate with a base.
